(19) United States
(12) Patent Application Publication
(10) Pub. No.: US 2025/0381160 A1
(43) Pub. Date: Dec. 18, 2025

(54) Method and Apparatus for Providing Transdermal Delivery of a Carbidopa/Levodopa-Based Lotion for the Treatment of Parkinson's Disease

(71) Applicant: Through The Skin LLC, Centennial, CO (US)

(72) Inventors: Christopher William Hovey, Centennial, CO (US); Brian Stephan Lukian, Toronto (CA)

(21) Appl. No.: 19/238,833

(22) Filed: Jun. 16, 2025

Related U.S. Application Data
(60) Provisional application No. 63/660,461, filed on Jun. 14, 2024.

(51) Int. Cl. A61K 31/198; A61K 9/00; A61K 9/06; A61K 47/36

(52) U.S. Cl. CPC A61K 31/198; A61K 9/0014; A61K 9/06; A61K 47/36

(57) ABSTRACT

Disclosed is a system and method for a non-oral dopaminergic therapy for patients with Parkinson's disease, the administration thereof having the benefit of no change in risk to the patient. The disclosed therapy is capable of delivering prescribed doses of carbidopa/levodopa through the skin into the bloodstream where it may circulate systemically in the body, bypassing the digestive system and avoiding first-pass metabolism in the liver. The dislosed drug delivery system enables therapeutic dosages of carbidopa/levodopa to reach the target tissue site in a controlled manner while maintaining the concentration in the therapeutic window.

### FIELD OF ART

The disclosed method and apparatus relate generally to a treatment for symptoms of Parkinson's Disease and its syndrome (Parkinsonism) and more specifically to a mechanism for the transdermal delivery to a patient of one or more doses of carbidopa 25 mg/levodopa 100 mg and a method therefor.

### BACKGROUND

Parkinson's Disease (PD) is a progressive neurodegenerative disorder of the extrapyramidal nervous system affecting the mobility and control of the skeletal muscular system. Symptoms of PD include tremors or shaking, stiffness, imbalance and bradykinesia (slowness of movement). Current evidence indicates that symptoms of PD are related to a lack of, or depletion of, dopamine, a natural substance usually found in the brain. Dopamine helps provide control of movement and activities such as walking, talking, dressing and handling objects, etc. The primary drugs for initial treatment of PD are levodopa and the dopamine agonists.

PD most often presents after age 60 and patients in this age group are best managed with levodopa therapy as the primary treatment modality. Unlike young-onset (<age 40) parkinsonism, the older age group is much less susceptible to the delayed motor complications of levodopa therapy, namely dyskinesias and motor fluctuations. When these problems do occur in seniors, however, they usually can be managed by medication adjustments. The treatment goal is to keep patients active and engaged. For the past four decades, the foundation of symptomatic treatment of PD has been levodopa, which continues to be the most efficacious medication for PD. As will be discussed further below, the typical route of administration of levodopa is by mouth.

Levodopa is in a class of medications called central nervous system agents. When levodopa was first introduced in the late 1960's, large doses were necessary to overcome its rapid degradation by dopa decarboxylase (DDC) which causes the conversion of levodopa to dopamine. DDC is a homodimeric, pyridoxal phosphate dependent enzyme that is located in different areas of the brain and is mostly found in the basal ganglia. This conversion of levodopa to dopamine occurs in the peripheral circulation as well as in the central nervous system after levodopa has crossed the blood-brain barrier. While the activation of central dopamine receptors improves the symptoms of PD, the dopamine generated in the circulation by peripheral DDC can cause nausea and vomiting. The addition of dopa decarboxylase inhibitor (DDCI) in the early 1970's to block the action of the decarboxylase was a huge advancement in the treatment of PD. Thus, levodopa is usually administered in combination with an inhibitor, in this case, carbidopa. Carbidopa does not cross the blood-brain barrier. It works by preventing levodopa from being broken down in the peripheral circulation before it reaches the brain, which reduces the nausea and vomiting often experienced by a patient. Preventing the breakdown of levodopa also increases the quantity of levodopa in the bloodstream that is available to enter the brain, thus allowing for a lower dose of levodopa. Levodopa is converted into dopamine in the brain and restores the depleted dopamine levels. Although non-levodopa-based treatment therapies such as rotigotine, apomorphine, etc. are available, carbidopa/levodopa is the drug that is primarily used to manage the symptoms of PD. Carbidopa/levodopa is listed on the World Health Organization's List of Essential Medicines and is sold under several brand names, including Sinemet® (Organon NV), Rytary® (Amneal Pharmaceuticals LLC), Duopa (AbbVie AB), Apo (Apotex Inc). It is also available as a generic (known as levocarb or co-careldopa).

It should be noted that the two available DDC inhibitors, carbidopa and benserazide, are largely interchangeable with no apparent advantages of one over the other. As such, a conventional dosage of each is 25 mg, formulated with 100 mg of levodopa. The discussion of the disclosed transdermal delivery system however will focus on the formulation of carbidopa/levodopa but it should be noted that one having ordinary skill in the art will recognize benserazide/levodopa could be a suitable formulation option as it is dosed identically.

As stated above, the typical route of administration of carbidopa/levodopa is by mouth. The oral medication comes in many formulations and strengths, e.g., immediate-release tablets, extended-release (long-acting) tablets and capsules, orally disintegrating tablets, etc., but each must be taken multiple times a day in order to effectively relieve a patient's PD symptoms. For example, the regular and orally disintegrating tablets are usually taken three or four times a day. The extended-release tablet is usually taken two to four times a day. The extended-release capsule is usually taken three to five times a day. The long-duration effect from levodopa requires about a week to fully manifest and treatments may take up to two to three weeks before benefits are seen. In some cases, it may several months before a patient may feel the full effect of carbidopa/levodopa. During the initiation of treatment and with continued treatment, it can be recognized that a patient with PD will have ingested (or attempted to ingest) hundreds of pills orally. As further discussed below, it is recognized that patients with PD face challenges with orally administered medications.

Dysphagia, or difficulty in swallowing is extremely common for many people with PD. Although the underlying pathophysiology is not well understood, it has been shown that both dopaminergic and non-dopaminergic mechanisms are involved in the development of dysphagia in PD. Dysphagia can be caused when the muscles and nerves used for swallowing are damaged or weakened or when a health condition causes. the back of the throat or esophagus to narrow. In Parkinson's, rigidity and bradykinesia often cause dysphagia. The movement and swallowing issues experienced by patients with PD are caused by the loss of dopamine-producing neurons in the substantia nigra area of the brain. At the same time, loss of neurons in other areas of the brain can impact the overall control and coordination of swallowing, so dysphagia is sometimes considered both a non-motor and motor symptom. Not only does dysphagia lead to the inability or difficulty of swallowing food, liquids, and/or pills, patients can experience pain, coughing or gagging, regurgitation, the feeling that food is stuck in the throat or chest, heartburn, drooling, weight loss, respiratory problems and delayed gastric emptying. Studies show that more than 80% of people with PD will develop dysphagia at some point during the course of their disease.

Strategies for managing dysphagia can range from taking basic actions such as ensuring patients thoroughly chew food to undergoing more complex swallowing treatments by speech and language therapists. Severe situations could even involve surgical interventions such as deep brain stimulation. Dysphagia usually becomes more severe as the disease progresses.

Swallowing impairment not only complicates medicine intake and reduces quality of life, it can lead to aspiration pneumonia which is a major cause of death in PD. Thus, it is reasonable for patients with dysphagia to seek other drug forms or other drug options to avoid the oral consumption of a pill. For example, carbidopa/levodopa can be administered in liquid form as a suspension through a naso-jejunal tube inserted into the nose and down into the stomach. Another possible solution involves the direct subcutaneous delivery of a carbidopa/levodopa solution to a patient's small intestine via an infusion pump. In any case, the above-described issues experienced with oral ingestion of medication as well as those encountered with invasive alternate therapies could lead to other potentially serious problems.

It is also now known that gut dysfunction can affect all stages of PD. Gut abnormalities such as small intestinal bacterial overgrowth as well as altered gut microbiota can interfere with gastric absorption of oral therapies which can lead to symptoms of nausea, vomiting, “off” periods and motor and non-motor fluctuations. Off periods occur when PD medication, namely levodopa, is not working optimally and a patient's symptoms return. These symptoms can include motor symptoms, such as tremor and rigidity, and non-motor symptoms, such as anxiety. Often such problems can only be effectively managed by non-oral therapies that avoid the gastrointestinal system such as the aforementioned naso-jejunal and subcutaneous/levodopa infusional therapies, levodopa inhalant therapy, apomorphine infusionals, etc. each of which have treatment advantages and disadvantages. For example disadvantages of the transdermal rotigotine patch include the Federal Drug Administration's general concern regarding patches propensity to leave residual materials, the potential for a lack of uniformity of doses, and the prevalence of skin reactions which can complicate therapy.

By providing a transdermal delivery option for carbidopa/levodopa applicant's non-oral dopaminergic therapy serves a long-felt and unmet need in the industry. Applicant's system enables a patient to administer, with the guidance of the patient's physician, prescribed dosage(s) of the transdermally delivered medication without the oral ingestion of multiple dry tablets or capsules, thus lowering the ever-present discomfort of dysphagia and its attributable risks. In addition, Applicant's system provides for a reliable solution in view of the increasing recognition of the extent of multilevel gastrointestinal dysfunction in PD.

Applicant's system adds no additional risk to the patient as it does not change the prescribed dosage regimen. It simply provides the prescribed dosage(s) albeit in a more acceptable and desirable format. In addition, Applicant's system allows a patient and the patient's doctor to continue self-administration of carbidopa/levodopa dosage(s) to manage motor and non-motors aspects of PD without having to opt for non-levodopa-based therapies or infusion.

### SUMMARY OF THE DISCLOSURE

An aspect of the disclosed system is to provide for a non-oral dopaminergic therapy for patients with Parkinson's Disease.

Another aspect of Applicant's system is to provide a mechanism for the transdermal delivery to a patient of one or more doses of carbidopa 25 mg/levodopa 100 mg.

Another aspect of Applicant's system is to enable a patient to administer prescribed dosage(s) of transdermally delivered medication, with the guidance of the patient's physician, without the oral ingestion of multiple dry tablets or capsules.

Yet another aspect of the disclosed system is that it helps to lower a patient's discomfort of dysphagia and its attributable risks.

Another aspect of Applicant's system is to provide for a reliable solution in view of the increasing recognition of the extent of multilevel gastrointestinal dysfunction in PD.

An aspect of the disclosed system is that it does not change the prescribed dosage amount, and thereby adds no additional risk to the patient as it does not change the prescribed dosage regimen.

Another aspect of the disclosed system is that it provides the prescribed dosage(s) in a more acceptable and desirable format.

Yet another aspect of the disclosed system is that it allows a patient to continue self-administration of carbidopa/levodopa dosage(s) to manage motor and non-motors aspects of PD without having to opt for non-levodopa-based therapies or infusion.

Yet another aspect of Applicant's system is that it allows for prescribed doses of carbidopa/levodopa to pass into the bloodstream through the skin where it may circulate systemically in the body, bypassing the digestive system and avoiding first-pass metabolism in the liver.

Yet another aspect of Applicant's system is that it is non-invasive and can be started or stopped at any time.

Another aspect of Applicant's system is that it enables therapeutic dosages of carbidopa/levodopa to reach the target tissue site in a controlled manner while maintaining the concentration in the therapeutic window.

These and other advantages of the disclosed device will appear from the following description and/or appended claims, reference being made to any accompanying drawings that form a part of this specification. It is to be understood that the disclosed method and apparatus are not limited in application to the details of the particular arrangements shown; the disclosed method and apparatus each capable of other embodiments. Also, the terminology used herein is for the purpose of description and not of limitation.

### DESCRIPTION OF THE DISCLOSED EMBODIMENT

The following description is provided to enable any person skilled in the art to make and use the disclosed system and method. Various modifications, however, will remain readily apparent to those skilled in the art, since the generic principles of the present apparatus have been defined herein specifically to provide for transdermal delivery of a combination of carbidopa and levodopa for the treatment of Parkinson's Disease.

Carbidopa is an inhibitor of aromatic amino acid decarboxylation. It is a white, crystalline compound, slightly soluble in water, with a molecular weight of 244.3. It is designated chemically as (-)-L-α-hydrazino-α-methyl-β-(3,4-dihydroxybenzene) propanoic acid monohydrate. The empirical formula is C₁₀H₁₄N₂O₄·H₂O.

The structural formula is:

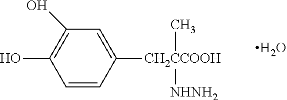

Tablet content is expressed in terms of anhydrous carbidopa which has a molecular weight of 226.3.

Levodopa is an aromatic amino acid. It is a white, crystalline compound, slightly soluble in water, with a molecular weight of 197.2. It is designated chemically as (-)-L-α-amino-β-(3,4-dihydroxybenzene) propanoic acid. The empirical formula is C₉H₁₁NO₄. The structural formula is:

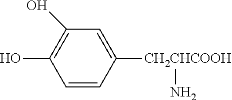

Various formulations of carbidopa/levodopa are known in the industry. For example, one combination of carbidopa/levodopa contains 25 mg of carbidopa and 100 mg of levodopa. Another combination of carbidopa/levodopa contains 10 mg of carbidopa and 100 mg of levodopa. Yet another combination of carbidopa/levodopa contains 25 mg of carbidopa and 250 mg of levodopa. Each contains inactive ingredients, i.e., microcrystalline cellulose, pregelatinized starch, starch (corn), and magnesium stearate. The 25 mg carbidopa/100 mg levodopa combination also contains D&C Yellow #10. The 10 mg carbidopa/100 mg levodopa combination and the 25 mg carbidopa/100 mg levodopa combination also contain FD&C Blue #2.

Applicant has developed a system for the transdermal delivery of a drug treatment containing 25 mg carbidopa and 100 mg levodopa which is an immediate-release formulation of carbidopa-levodopa that begins release of ingredients within about 30 minutes. It is to be understood that the disclosed system could be implemented using other formulations, concentrations and ratios of carbidopa/levodopa if desired. For example, one having ordinary skill in the art could modify the disclosed system to implement formulations containing 25 mg carbidopa and 250 mg levodopa. Furthermore, it may be suitable to use benserazide/levodopa formulations instead of carbidopa/levodopa formulations as described above. Nonetheless, the discussion of the disclosed transdermal delivery system will focus on an embodiment comprising a formulation of 25 mg carbidopa and 100 mg levodopa (e.g., Sinemet 25-100), hereinafter “the 25-100 formulation”. Such tablets are yellow oval tablets containing 25 mg carbidopa and 100 mg levodopa. They appear plain on one side, while the other side exhibits a “650” and a score line.

When levodopa is administered orally, it is quickly decarboxylated to dopamine outside of the brain so that only a small portion of a given dose is transported unchanged to the central nervous system. For this reason, large doses of levodopa are required for adequate therapeutic effect. As stated above, nausea and other adverse reactions can be attributed to dopamine formed in extracerebral tissue.

Carbidopa inhibits decarboxylation of peripheral levodopa. Since its decarboxylase inhibiting activity is limited to extracerebral tissues, administration of carbidopa with levodopa makes more levodopa available for transport to the brain. It does not affect the metabolism of levodopa within the central nervous system since it does not cross the blood-brain barrier. In many patients, the resulting reduction in nausea and vomiting using carbidopa will permit more rapid dosage titration.

Furthermore, carbidopa reduces the amount of levodopa required to produce a given response by about 75% and, when administered with levodopa, increases both plasma levels and the plasma half-life of levodopa and decreases plasma and urinary dopamine and homovanillic acid. Without carbidopa, the plasma half-life of levodopa is about 50 minutes. When carbidopa and levodopa are administered together, the half-life of levodopa is increased to about 1.5 hours. At steady state, the bioavailability of carbidopa from the 25-100 formulation is approximately 99% relative to the concomitant administration of carbidopa and levodopa.

In addition to the carbidopa/levodopa treatment itself, patients with PD are often advised to take Vitamin B-6 (pyridoxine hydrochloride or “pyridoxine”) which is important for normal brain development and for keeping the nervous system and immune system healthy. Pyridoxine in oral doses of 10 mg to 25 mg may reverse the effects of levodopa by increasing the rate of aromatic amino acid decarboxylation. Carbidopa however inhibits this action of pyridoxine which allows for the 25-100 formulation described herein to be given to patients receiving supplemental pyridoxine without reducing the effectiveness of the medications.

It can be appreciated that the optimum daily dosage of a carbidopa/levodopa formulation must be determined by careful titration in each patient. However, as discussed herein, the 25-100 formulation is available in a 1:4 ratio of carbidopa to levodopa. A sample regimen is initiated with one tablet of the 25-100 formulation three times a day. The dosage schedule thus provides 75 mg of carbidopa per day. It is noted that each dose can begin working in about ten minutes to two hours with a duration of effect of about five hours. Studies show that peripheral DDC is saturated by carbidopa at approximately 70 to 100 mg a day. Patients receiving less than this amount of carbidopa are more likely to experience nausea and vomiting. Therefore, dosage may be increased by one tablet of the 25-100 formulation every day or every other day, as necessary. It is important that the formulation be administered at regular intervals according to the schedule outlined by a patient's medical advisor.

The system disclosed herein can deliver medication directly into the bloodstream of a patient through the layers of the skin at a prescribed rate. The system is non-invasive and can be started or stopped at any time. When applied to the skin, Applicant's system can deliver active ingredients into a patient's systemic circulation via diffusion. Applicant's drug delivery system enables therapeutic dosages of carbidopa/levodopa to reach the target tissue site in a controlled manner while maintaining the concentration in the therapeutic window.

Applicant's system uses a bioactive material that elicits a specific biological response at the interface of the skin. One embodiment disclosed herein employs the use of bioactive glass (or Bioglass) comprising calcium sodium phosphosilicate as it has been shown to promote vascularization and accelerate wound healing. Not only does Bioglass keep the application sites free from infection, it is inert, biologically compatible with the carbidopa/levodopa, controls irritation at the application site, and has the ability to carry high doses of the drug with no risk of accidental release.

Applicant's system uses a fraction of medical grade hyaluronic acid (HA) as a carrier for the carbidopa/levodopa and Bioglass formulation, wherein the molecular weight of the HA is between about 150,000 to about 750,000 Daltons. Hyaluronic acid has good biocompatibility, biodegradability, and non immunogenicity, however, the standard hyaluronic acid (HA) molecule at an average molecular weight of 7 million Daltons lacks the ability to permeate the skin, and when topical HA is applied to the skin, it remains on the skin's surface and functions as a skin-surface moisturizer. Applicant's system uses a specific fraction of medical grade HA as the transdermal delivery vehicle for carbidopa/levodopa.

U.S. Pat. No. 5,639,738 purports to teach a treatment of basal cell carcinoma and actinic keratosis using HA and NSAIDS, specifically diclofenac, an arthritis drug with a molecular weight of 296.148 g/mol. Diclofenac sodium has a molecular weight of 318.129 g/mol. It was found that when used in the topical treatment of actinic keratosis, the specific fraction of the HA molecule having a mean average molecular weight between 150,000 and 750,000 Daltons was shown to increase by 3 times the amount of diclofenac sodium that penetrated the skin compared to formulations and combinations of diclofenac sodium not containing hyaluronic acid. The concentration of the HA used was between 1-3% of the solution.

Carbidopa has a molecular weight of 226.229 g/mol. Levodopa has a molecular weight of 197.188 g/mol. It is contemplated that since each has a molecular weight close to about 296.148 g/mol, the HA will similarly be able to carry the carbidopa/levodopa formulation through the skin. The concentration of the HA in Applicant's system is between about 1-2% by weight of the composition.

Applicant has reported positive results in a series of preliminary trials. In one trial, a patient diagnosed with Parkinson's Disease had undergone a daily 6-pill regimen of carbidopa 25 mg/levodopa 100 mg (2 pills 3 times a day) for 13 years. As the Parkinson's progressed, the patient's treatment was increased to a 12-pill regimen of carbidopa 25 mg/levodopa 100 mg (2 pills 6 times per day) in addition to other medications in pill form which were prescribed to help increase the efficiency of the limited dopamine suffered by the patient. In sum, the patient was orally taking 15 pills a day: 13 carbidopa 25 mg/levodopa 100 mg, 1 Rasagiline 1 mg and 1 Ongentiys 25 mg. Not only was the patient experiencing dysphagia, it was noted that at the time of the trial, the patient was at the upper limit of acceptable amounts of carbidopa/levodopa as prescribed. The patient was therefore forced to consider other forms of treatment, being virtually assured of continued discomfort and facing the risks brought on by dysphagia.

Because the alternatives to carbidopa 25 mg/levodopa 100 mg were not appealing, the patient undertook the following steps in the trial. The patient's daily pill regimen consisted of taking 2 carbidopa 25 mg/levodopa 100 mg pills at the prescribed times: at 7:00 am, 10:00 am, 1:00 pm, 4:00 pm, 7:00 pm and 10:00 pm. It was noted the patient experienced off periods at 4:00 pm, during which the medication was less effective and symptoms returned. During the trial, the patient continued to orally take 2 carbidopa 25 mg/levodopa 100 mg pills at the prescribed times of 7:00 am, 10:00 am, 1:00 pm, 7:00 pm and10:00 pm. For the 4:00 pm dosage however, the medication was administered topically. In short, the typical oral dose of 2 carbidopa 25 mg/levodopa 100 mg pills was changed into a paste suitable for transdermal delivery whereby the 2 individual carbidopa 25 mg/levodopa 100 mg pills were ground into a fine powder via in a pharmaceutical mortar and pestle suited to retain the entirety of the dosage and mixed with medical grade hyaluronic acid that had been premixed with pharmaceutical water and Bioglass to make a light paste. The patient administered the totality of the paste directly from the mortar to the chest by hand. After about 15 minutes, it was noted that the patient could feel the medicine starting to work and after about 45 minutes, the patient reportedly was in a full “on” period. Thus for the 4:00 pm dosage, it was observed that the transdermal method of treatment yielded results approximately 15 minutes faster than what was typically experienced when the dosage was taken orally. In addition, it was reported that the “on” period lasted longer when the dosage was administered transdermally versus orally. Subsequent tests were conducted on the patient and the results consistently confirmed the findings. The subsequent trials also demonstrated the potential feasibility of transdermal delivery of carbidopa 25 mg/levodopa 100 mg as an alternative to oral therapy, thereby allowing a patient to avoid swallowing problems.

Applicant's drug delivery system is capable of providing controlled drug delivery without the risk of increased or decreased acceptance. Applicant's system comprises a specially prepared formulation of carbidopa 25 mg/levodopa 100 mg housed in a convenient applicator. It is a precisely formulated carbidopa/levodopa-based lotion designed for topical administration and contains the same number of pill equivalents as that in the raw material entering the process and can be directly applied to a patient's skin according to a prescribed dosage schedule. This formulation provides a convenient dosage form for accurate transdermal delivery of medication and is particularly beneficial for patients who find it difficult to swallow solid dosage forms or who may require a more manageable form of medication. The medication should be stored in a tightly closed container at room temperature, away from high heat, moisture, direct light, and freezing.

The input for the process disclosed herein comprises a specified number of dry tablets received from a source. The output of the process disclosed herein comprises said specified number of dry tablets in an alternate form for delivery to a customer. Thus, the process disclosed herein is capable of delivering the exact number of doses transdermally as the specified number of dry tablets available orally. In short, one dose equates with one dry tablet. The process is a closed-loop system, which helps to ensure the end-user patient will not receive an excess or a deficiency in dosages of carbidopa/levodopa which in pill count is tracked regardless of the size of the order. For example, one batch could be a single order (e.g., one dry tablet, 30 dry tablets, etc.), several orders or a time-based production (e.g., 8 hour, 24 hour, etc.).

When an order is received for a specific number of doses, a purchase order will be generated for a corresponding number of carbidopa 25 mg/levodopa 100 mg finished tablets from a pre-approved source. The process begins with the delivery of the requisite number of finished tablets, or raw material, pursuant to the purchase order and the confirmation of the number of tablets received by the receiving pill counter.

Applicant's system utilizes an Automatic Electronic Pill Counting Machine CDR-3A with conveyor which has a capacity of 1300 tablets per minute. Applicant's system ensures that the doses emerging at the end of the closed-loop process comprises the specific number of tablets entering the process.

For each batch, a finite quantity of incoming dry tablets is ground into a powder having a particle size of 100 microns using a fine grinder. The powder is then mixed with a premixed liquid fraction of medical grade HA with a molecular weight of 150,000 daltons (grams per mole) to 750,000 daltons, combined with bioglass having a particle size of 100 microns and pharmaceutical water in an in-line mixer, such that the resultant mixture comprises 2.5% HA, 3% C/L, 2.5% bioactive glass and 92% pharmaceutical water.

It will be recognized by one having ordinary skill in the art that various grinders, inline mixers and other suitable production equipment may be used depending on the size and scope of the process. In one configuration, a Cecile Ultra grinder and a Rotor Stator Mixer were employed. It is contemplated that pharmaceutical water could be generated on-site, however, the examples in the discussion herein utilize packaged pharmaceutical waters, which are produced, packaged, and sterilized to preserve microbial quality throughout the packaged shelf life.

Employing one or more benchtop fillers, the resultant doses are packaged into an appropriately sized applicator that is capable of reliably dispensing an accurate dose. The applicators are shipped back to a client drug company for ultimate dispatch to end users. In operation, a patient applies the mixture which comprises carbidopa/levodopa suspended in liquid HA/bioglass to the skin in an accurate prescribed amount. A patient may choose to dispense a pre-measured dose from the applicator on to their skin, e.g., chest, arm, etc., and then rub it in.

Many carbidopa/levodopa users take medication in 2-pill increments. Thus, in one embodiment, single or two-pill doses are packaged in syringe-style applicators. Some carbidopa/levodopa users take as many as 15 pills per day. A 100-pill applicator is available for such applications. It should be understood that applications in other configurations and volumes would fall within the scope of this disclosure.

It is contemplated that the production equipment, facility and equipment surfaces and all containers be sprayed with a vegetarian version of LiquiGlide to create durable and slippery surfaces so as to reduce the likelihood of waste or residual product and to ensure the end-user patient will not receive an excess or deficient dosages of carbidopa/levodopa. This step helps to ensure that the pill input and dosage output achieve a 100% match.

As discussed above, Applicant's system has the potential of addressing dysphagia as well as multilevel gastrointestinal dysfunction in people with PD. Furthermore, applicant believes the process disclosed herein can be applied to the transdermal delivery of any drug that comes in dry tablet or pill form where the leading active drug has a molecular weight less than about 500.00 g/mol.

Applicant discloses a transdermal drug delivery system for treating the symptoms of PD, the system comprising one or more doses of carbidopa 25 mg/levodopa 100 mg finely ground into a powder having a mesh size of about 100 microns, a hyaluronic acid in an amount effective to transport the one or more doses of carbidopa 25 mg/levodopa 100 mg transdermally at a site of application by a patient, the hyaluronic acid being premixed with pharmaceutical water and a bioactive glass and combined with the powder to form a low viscosity carbidopa/levodopa-based paste or lotion. The hyaluronic acid comprises a molecular weight between about 150,000 Daltons and about 750,000 Daltons. In addition, the hyaluronic acid comprises a concentration of about 1-2.5% by weight of the composition. The paste or lotion is housed in an applicator capable of dispensing at a controlled rate the carbidopa/levodopa-based lotion housed therein. The paste or lotion further comprises about 3% carbidopa-levodopa, about 2.5% bioactive glass and about 92% pharmaceutical water.

Applicant disloses a method of providing a dispenser containing an equivalent of one more doses of carbidopa 25 mg/levodopa 100 mg in dry tablet form. The method comprises the steps of: determining a requisite batch size, using as a feedstock, one or more carbidopa 25 mg/levodopa 100 mg dry tablets from an approved source, confirming the requisite batch size by processing said feedstock through a tabulation device to obtain a definitive input value which can be qualitatively compared to a process output, grinding said tabulated feedstock to produce a powder having a mesh size of about 100 microns, mixing said powder with a premixed solution comprising medical grade hyaluronic acid combined with pharmaceutical water and bioglass, thereby forming a low viscosity paste comprising a formulation of carbidopa 25 mg/levodopa 100 mg equivalent to said definitive input value, and filling one of more dispensers with said equivalent formulation, thereby allowing for a topical application of said equivalent formulation by a patient at a controlled rate. The medical grade hyaluronic acid comprises a molecular weight between about 150,000 Daltons and about 750,000 Daltons. The method further comprises the step of applying a slippery surface technology to one or more surfaces in contact with carbidopa 25 mg/levodopa 100 mg in all its forms to ensure said process output correlates closely with said definitive input value.

Applicant discloses a method of topically administering an effective prescribed dosage of carbidopa/levodopa one or more times a day on at least one surface of a patient's skin for a duration sufficient to treat symptoms associated with Parkinson's Disease or parkinsonism.

Although the disclosed device and method have been described with reference to disclosed embodiments, numerous modifications and variations can be made and still the result will come within the scope of the disclosure. No limitation with respect to the specific embodiments disclosed herein is intended or should be inferred.

We claim:

1. A transdermal drug delivery system for treating the symptoms of PD, the system comprising:
one or more doses of carbidopa 25 mg/levodopa 100 mg finely ground into a powder having a mesh size of about 100 microns;
a hyaluronic acid in an amount effective to transport the one or more doses of carbidopa 25 mg/levodopa 100 mg transdermally at a site of application by a patient; the hyaluronic acid being premixed with pharmaceutical water and a bioactive glass and combined with the powder to form a low viscosity carbidopa/levodopa-based paste or lotion;
the hyaluronic acid having a molecular weight between about 150,000 Daltons and about 750,000 Daltons;
the hyaluronic acid in a concentration of about 1-2.5% by weight of the composition;
the paste or lotion being housed in an applicator capable of dispensing at a controlled rate the carbidopa/levodopa-based lotion housed therein; and
wherein the paste or lotion further comprises about 3% carbidopa-levodopa, about 2.5% bioactive glass and about 92% pharmaceutical water.

2. A method of providing a dispenser containing an equivalent of one more doses of carbidopa 25 mg/levodopa 100 mg in dry tablet form, the method comprising the steps of:
determining a requisite batch size;
using as a feedstock, one or more carbidopa 25 mg/levodopa 100 mg dry tablets from an approved source;
confirming the requisite batch size by processing said feedstock through a tabulation device to obtain a definitive input value which can be qualitatively compared to a process output;
grinding said tabulated feedstock to produce a powder having a mesh size of about 100 microns;
mixing said powder with a premixed solution comprising medical grade hyaluronic acid combined with pharmaceutical water and bioglass, thereby forming a low viscosity paste comprising a formulation of carbidopa 25 mg/levodopa 100 mg equivalent to said definitive input value;
said medical grade hyaluronic acid having a molecular weight between about 150,000 Daltons and about 750,000 Daltons; and
filling one of more dispensers with said equivalent formulation, thereby allowing for a topical application of said equivalent formulation by a patient.

3. The method of claim 2, further comprising the step of applying a slippery surface technology to one or more surfaces in contact with carbidopa 25 mg/levodopa 100 mg in all its forms to ensure said process output correlates closely with said definitive input value.

4. A method of topically administering an effective prescribed dosage of carbidopa/levodopa one or more times a day on at least one surface of a patient's skin for a duration sufficient to treat symptoms associated with Parkinson's Disease or parkinsonism.
